(19) United States
(12) Patent Application Publication
(10) Pub. No.: US 2025/0200387 A1
(43) Pub. Date: Jun. 19, 2025

(54) OPTIMIZING LARGE LANGUAGE MODELS WITH META LEARNING AND CHAIN OF THOUGHT

(71) Applicant: NEC Laboratories America, Inc., Princeton, NJ (US)

(72) Inventors: Xujiang Zhao, Hillsborough, NJ (US); Haifeng Chen, West Windsor, NJ (US)

(21) Appl. No.: 18/975,717

(22) Filed: Dec. 10, 2024

Related U.S. Application Data
(60) Provisional application No. 63/609,929, filed on Dec. 14, 2023.

(51) Int. Cl. G06N 3/0985; G16H 50/20

(52) U.S. Cl. CPC G06N 3/0985; G16H 50/20

(57) ABSTRACT

Systems and methods for optimizing large language models with meta learning and chain of thought. A large language model (LLM) can be fine-tuned by generating optimized prompts based on an associated score of a generated output relative to a target output of a LLM-based optimizer using tuples of questions and the prompts generated from a dataset. Core features from the dataset that obtained top-ranked associated scores for the optimized prompts can be learned by utilizing chain of thought mechanism with the LLM-based optimizer. A meta prompt from the core features can be generated with the LLM-based optimizer to perform downstream tasks with the LLM.

### RELATED APPLICATION INFORMATION

This application claims priority to U.S. Provisional App. No. 63/609,929, filed on Dec. 14, 2023, incorporated herein by reference in its entirety.

### BACKGROUND

### Technical Field

The present invention relates to training artificial intelligence (AI) models and more particularly to optimizing large language models with meta learning and chain of thought.

### Description of the Related Art

Large language models (LLM) have advanced to powerful tools capable of advanced natural language processing tasks such as text summarization, question answering, and relevant text generation. However, the accuracy and efficiency of the outputs of LLMs are tied to its inputs. An ineffective input for an LLM would drastically lower the accuracy and efficiency of the outputs of the LLM. Thus, enhancing the accuracy of the inputs would also enhance the accuracy of the output of the LLM.

### SUMMARY

According to an aspect of the present invention, a computer-implemented method for optimizing artificial intelligence (AI) models is provided, including, fine-tuning a large language model (LLM) by generating optimized prompts based on an associated score of a generated output relative to a target output of a LLM-based optimizer using tuples of questions and prompts generated from a dataset, learning core features from the dataset that obtained top-ranked associated scores for the optimized prompts by utilizing chain of thought mechanism, and generating a meta prompt from the core features with the LLM-based optimizer global learning optimizer to perform downstream tasks with the LLM.

According to another aspect of the present invention, a system for optimizing artificial intelligence (AI) models is provided including, a memory device, and one or more processor devices operatively coupled with the memory device to, fine-tune a large language model (LLM) by generating optimized prompts based on an associated score of a generated output relative to a target output of a LLM-based optimizer using tuples of questions and prompts generated from a dataset, learn core features from the dataset that obtained top-ranked associated scores for the optimized prompts by utilizing chain of thought mechanism, and generate a meta prompt from the core features with the LLM-based optimizer global learning optimizer to perform downstream tasks with the LLM.

According to yet another aspect of the present invention, a non-transitory computer program product is provided including a computer readable storage medium having program code for optimizing artificial intelligence (AI) models, wherein the program code when executed on a computer causes the computer to fine-tune a large language model (LLM) by generating optimized prompts based on an associated score of a generated output relative to a target output of a LLM-based optimizer using tuples of questions and prompts generated from a dataset, learn core features from the dataset that obtained top-ranked associated scores for the optimized prompts by utilizing chain of thought mechanism, and generate a meta prompt from the core features with the LLM-based optimizer global learning optimizer to perform downstream tasks with the LLM.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a flow diagram illustrating a high-level overview of a computer-implemented method for optimizing large language models (LLMs) with meta learning and chain of thought, in accordance with an embodiment of the present invention;

FIG. 2 is a block diagram showing a system implementing optimizing LLMs using meta learning and chain of thought to perform downstream tasks, in accordance with an embodiment of the present invention, in accordance with an embodiment of the present invention; and

FIG. 3 is a block diagram showing a computing device implementing optimizing LLMs using meta learning and chain of thought, in accordance with an embodiment of the present invention;

FIG. 4 is a block diagram showing hardware and software components employed to perform the operations in optimizing LLMs with meta learning and chain of thought, in accordance with an embodiment of the present invention; and

FIG. 5 is a block diagram showing a structure of deep neural networks for optimizing LLMs with meta learning and chain of thought, in accordance with an embodiment of the present invention.

### DETAILED DESCRIPTION OF PREFERRED EMBODIMENTS

In accordance with embodiments of the present invention, systems and methods are provided for optimizing large language models with meta learning and chain of thought.

In an embodiment, a large language model (LLM) can be fine-tuned by generating optimized prompts based on an associated score of a generated output relative to a target output of a LLM-based optimizer using tuples of questions and the prompts generated from a dataset. Core features from the dataset that obtained top-ranked associated scores for the optimized prompts can be learned by utilizing chain of thought mechanism with the LLM-based optimizer. A meta prompt from the core features can be generated with the LLM-based optimizer to perform downstream tasks with the LLM.

In recent years, artificial intelligence has witnessed remarkable advancements, giving rise to the emergence of large language models (LLMs) such as ChatGPT™ and Llama™. These LLMs have showcased their immense capabilities across various natural language processing (NLP) tasks. However, it is crucial to recognize that the performance of these LLMs is intricately tied to the quality of the prompts they receive. Extensive research has shown that when LLMs are provided with low-quality prompts, their performance can suffer, leading to undesirable behaviors and even the generation of harmful content. This issue becomes particularly worrisome when LLMs are deployed in safety-sensitive applications, where the consequences of inappropriate prompts can be significantly detrimental. Hence, it is evident that improving the quality of prompts is paramount in harnessing the full potential of LLMs while mitigating the associated risks.

To enhance the quality of prompts, manual prompt crafting can be employed. However, this method can be limited by the lack of expertise of users creating the prompts. Automated prompt optimization is another way of enhancing the quality of prompts. For white-box models like Llama™, gradient-based techniques are employed to adjust the prompt. In contrast, black-box models like ChatGPT™ pose a greater challenge due to the limited information available. Recent studies have tackled prompt optimization in black-box models using techniques that do not rely on gradient information, such as evolutionary algorithms. However, these methods encounter challenges, including performance degradation when faced with previously unseen prompts, and are highly dependent on the sequence of optimizing known prompts, resulting in an imbalanced emphasis on samples optimized later in the sequence.

To solve these challenges, the present embodiments present a framework to utilize an LLM-based optimizer with chain-of-thought and meta learning mechanism to optimize the prompts. By doing so, the present embodiments enhanced the performance of LLMs in performing downstream tasks such as toxicity reduction (e.g., removing bias or negative information from generated outputs), news summarization, and sentence simplification by as much as 25% in accuracy and efficiency scores.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

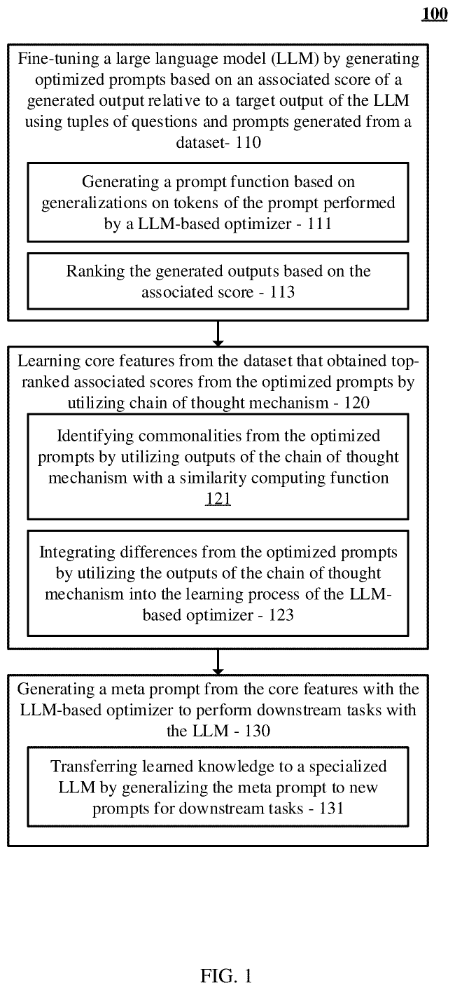

Referring now in detail to the figures in which like numerals represent the same or similar elements and initially to FIG. 1, a high-level overview of a computer-implemented method for optimizing large language models with meta learning and chain of thought, is illustratively depicted in accordance with one embodiment of the present invention.

In an embodiment, a large language model (LLM) can be fine-tuned by generating optimized prompts based on an associated score of a generated output relative to a target output of a LLM-based optimizer using tuples of questions and the prompts generated from a dataset. Core features from the dataset that obtained top-ranked associated scores for the optimized prompts can be learned by utilizing chain of thought mechanism with the LLM-based optimizer. A meta prompt from the core features can be generated with the LLM-based optimizer to perform downstream tasks with the LLM.

In block 110, a large language model (LLM) can be fine-tuned by generating optimized prompts based on an associated score of a generated output relative to a target output of a LLM-based optimizer using tuples of questions and the prompts generated from a dataset.

The question can be obtained from a dataset. The questions can remain unchanged (e.g., fixed) throughout the optimization process. The prompts are related to the question and can be obtained from the dataset. The prompts can serve as a system prompt that describes the characteristics (e.g., settings, output, input, etc.) of the LLM. The prompt can be provided by a user to instruct or guide the LLM in performing specific tasks. The prompts are optimized by the LLM. The tuples include the question and the prompt. In another embodiment, the questions and prompts are obtained from a database and are grouped together in tuples based on a determined relevance by a LLM-based text optimizer.

The dataset can be specific to the downstream tasks that are being performed. For example, for summarization tasks, a summarization dataset can be employed. The dataset can include questions, answers for the questions, other relevant information about the subject matter of the summarization task (e.g., medical data for medical text summarization tasks).

The tuples are fed to an LLM and the generated output of the LLM is processed with a target output. The target output can be obtained from the dataset. For example, for a medical data summarization task for a patient having respiratory issues with symptoms fever, cough, and body aches, the question can be “What symptoms does the patient have?,” the prompt can be “The patient has”, the generated output can include “a sickness.”, while the target output can include “fever, cough, and body aches.” In this example, the prompt function can include “<definite article> <subject> <action verb>”.

In block 111, the prompt function can be generated by the LLM-optimizer based on generalizations on tokens of the prompt performed by the LLM-optimizer.

In block 113, based on an optimization history, the tuples of associated scores and prompts are ranked based on the associated scores.

The associated score can include a relevance score depending on the downstream tasks being performed. The associated scores can be predefined in a database. For example, the associated score for medical text summarization can include a score function for computing content-similarity based relevance such as recall-oriented understudy for gisting evaluation (ROUGE) metrics, cosine similarity or embeddings, etc. In another example, the associated score for classification tasks can include a feature importance score computed using tree-based models such as random forest, shapely additive explanations (SHAP) values, etc.

To optimize the prompts, an LLM-based can be utilized for individual prompt optimization in black-box target LLM. The LLM can employ ChatGPT™, LLAMA™, etc. The optimization problem can be addressed as an optimization problem having a goal of achieving the highest possible score (p*) for each response generated by the LLMs:

$${*}^{} = arg {max}_{p} {E}_{q ∼ D} [ {f}_{sc} ( {L}_{ta} ( p , q ) ) ]$$

where q is a question obtained from a training dataset D, and p is prompt obtained from the training dataset D, f_{sc} is the score function associated with the downstream task, and Lₜₐ is the target output when given a prompt.

The goal of the optimization problem is to find a prompt function for generating prompts based on questions and the optimization history h. The optimization history can include tuples of an associated score and the prompt (p, s).

The prompt ( [symbol] ₜₒₚ) and the associated data (e.g., question, prompt from the dataset, relevant data from the dataset based on the downstream task performed, associated prompt function) from the top ranked tuple will be subjected to the LLM-based optimizer (Lₒ [symbol] ) to generate the optimized prompt ( [symbol] ₒₚ) with the following: [symbol] ₒ [symbol] =Lₒₚ (pₜₒₚ).

For example, a prompt included in the top-ranked tuple for medical text summarization can include: “A 50-year-old male with fever, cough, and shortness of breath, recently returned from overseas travel.” After optimization, the optimized prompt can include: “A 50-year-old male with fever (38.5° C.), dry cough, shortness of breath, returned from Southeast Asia 14 days ago, has a history of hypertension.”

In block 120, core features from the dataset that obtained high associated score from the optimized prompts can be learned by utilizing chain of thought mechanism with the LLM-based optimizer.

The present embodiments can utilize meta learning to learn the core features from the dataset which includes information on the relationship of an effective output and an optimized prompt. In meta learning, AI models can be trained to understand and adapt to new tasks on their own by generalizing across various tasks. In another embodiment, the present embodiments can utilize meta prompting to determine the optimized prompts. In meta prompting, a more abstract and structured way of interacting with LLMs is performed which emphasizes the form and pattern of information which allows the LLMs to focus on the structural and syntactic aspects of tasks and problems.

The core features can include features extracted from the associated data of the optimized prompts. The core features can include the generalizations of the associated data of the optimized prompts. For example, in the medical text summarization task example described herein, the core features can include temperature data, family medical history, travel history, respiratory illness symptoms, etc.

The chain of thought (CoT) mechanism can be employed to extract the core features. The CoT mechanism is a reasoning approach that explicitly breaks down a reasoning process into sequential steps. The CoT mechanism mimics how humans solve problems by thinking logically in a sequential manner.

For example, in the medical text summarization task example described herein with the following prompt: “A 50-year-old male with fever, cough, and shortness of breath, recently returned from overseas travel,” the chain of thought mechanism performed by the LLM-based global optimizer can include: “The patient is a 50-year-old male. The patient has an observed temperature of 38.5 degrees Celsius. The temperature range for a low-grade fever for a 50-year-old male generally ranges from 37.3 degrees to 38 degrees Celsius. The temperature range for a typical fever for a 50-year-old male generally ranges above 38 degrees Celsius. Then, the 50-year-old male has a fever because his temperature of 38.5 degrees is above 38 degrees.” The core features for this example can include the observed temperature of the patient and the temperature range for fever. The core features can be stored in a database and learned by the LLM-based global optimizer.

The core features can be learned through global learning, which analyze optimized individual prompt results and the associated scores, mitigating the negative effects caused by inappropriate optimization sequences and improving robustness. A global-learning LLM-based optimizer is employed to summarize the intrinsic features shared by high-scoring individual prompt results obtained during the individual prompt optimization stage. The global learning approach can utilize a chain-of-thought mechanism to unearth deeply hidden features, further enhancing the trustworthiness and robustness of L2P.

In block 121, commonalities between the associated data for the optimized prompts can be identified by utilizing outputs of the chain of thought mechanism with a similarity computing function.

To learn the core features for the optimized prompts, commonalities between the associated data for the optimized prompts can be identified. The commonalities can be determined by using similarity-based methods such as bag-of-words similarity, Jaccard similarity or semantic similarity which can compute a commonality score.

In block 122, differences from the optimized prompts can be integrated by utilizing the outputs of the chain of thought mechanism into the learning process of the LLM-based optimizer.

Additionally, to learn the core features for the optimized prompts, the differences between the optimized prompts can be identified and integrated. To identify the differences between the optimized prompts, various dimensions of the prompts can be compared that includes the intent, context, explicitness, scope, structure, clarity, etc., by using semantic similarity, the associated score and the score function.

For example, an optimized prompt A can include the following text: “A 50-year-old male with fever (38.5° C.), dry cough, shortness of breath, returned from Southeast Asia 14 days ago, has a history of hypertension.” An optimized prompt B can include the following text: “A 30-year-old female does 20-minute treadmill exercises twice a week.” An identified context for optimized prompt A can include symptoms of respiratory illness while an identified context for optimized prompt B can include lifestyle of a patient. The identified contexts between optimized prompt A and B can be integrated into the learning process of the LLM-based global optimizer.

In block 130, a meta prompt from the core features can be generated with a global learning optimizer to perform downstream tasks with the LLM.

The meta prompt can include the learned core features from the optimized prompts. The meta prompt can include the learned information on how the prompt functions for the optimized prompts are generated by the LLM-based optimizer. The meta prompt can include guided instructions on how the LLM can perform the downstream tasks based on the information provided.

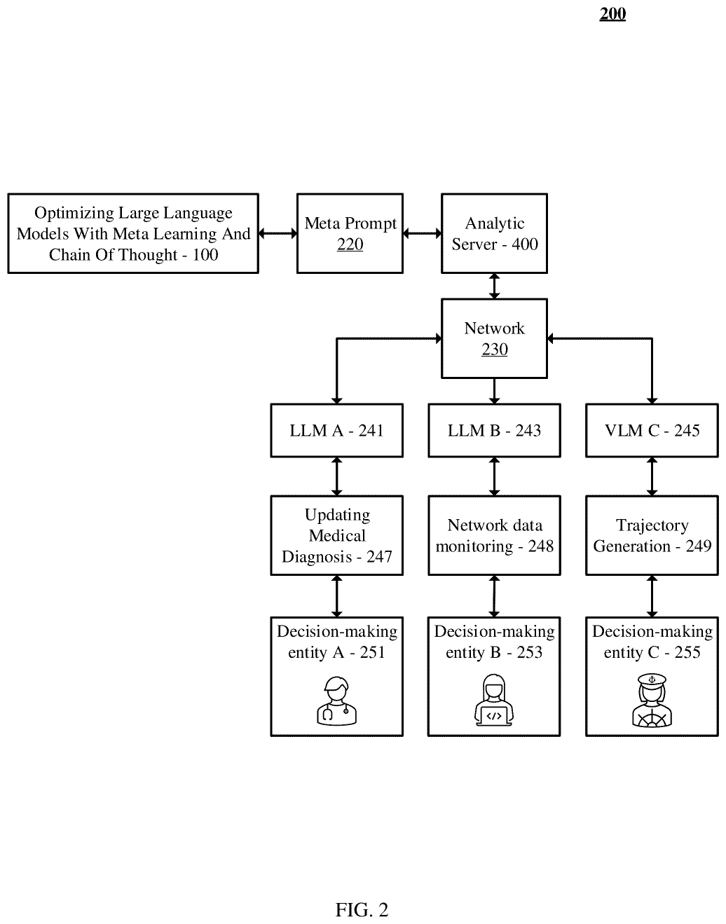

In block 131, learned knowledge can be transferred to a specialized LLM by generalizing the meta prompt to new prompts for downstream tasks. The meta prompt can be adapted and generalized for new prompts for various downstream tasks by transferring the learned knowledge of the LLM-based optimizer to specialized LLMs configured to perform various downstream tasks. This is shown in more detail in FIG. 2.

Referring now to FIG. 2, a block diagram showing a system implementing optimizing LLMs using meta learning and chain of thought to perform downstream tasks, in accordance with an embodiment of the present invention.

The system 200 can include an analytic server 240 that can implement optimizing large language models with meta learning and chain of thought 100 to generate a meta prompt 220 to perform various downstream tasks such as updating medical diagnosis 247, network data monitoring 248 and trajectory generation 249. The analytic server 240 can communicate with a network 230 to send and receive requests from computing nodes that communicate with decision-making entities.

Due to the adaptability of meta prompt 220, it can be used for different artificial intelligence models that is configured for the various downstream tasks. For example, LLM A 241 can be configured to perform updating medical diagnosis 247. LLM B 243 can be configured to perform network data monitoring 248. Visual-language model (VLM) C 245 can be configured to perform trajectory generation 249 for autonomous vehicles.

In updating medical diagnosis 247, a decision-making entity A 251 (e.g., health professional) can provide medical information to LLM A 241. LLM A 241 can generate the meta prompt 220 with the provided medical information to generate medical information summaries (e.g., summarized portions of patient medical history, etc.), provide medical knowledge suggestions (e.g., temperature range for fever, blood glucose level for pre-diabetes, treatment procedures for a specific disease such as insulin for diabetes, bronchodilators such as albuterol for asthma, etc.) to update the medical diagnosis of a patient. The decision-making entity A 251 can then update the medical diagnosis of the patient based on the medical information summaries and medical knowledge suggestions provided by LLM A 241.

In network data monitoring 248, a decision-making entity B 253 (e.g. network administrator) can provide network data stream information to LLM B 243. LLM B 243 can generate the meta prompt 220 with the provided network data stream information to detect data anomalies such as increased utilization of an application server from an identified internet protocol (IP) address. LLM B 243 can then generate optimal commands for the detected data anomalies such as blocking data packets from the identified internet protocol address, increasing bandwidth to the server, etc. The decision-making entity B 253 can then approve the optimal commands to be applied to the application server.

In trajectory generation 249, a decision-making entity C 255 (e.g., driver, autonomous vehicle) can provide traffic scene information to VLM C 245. VLM C 245 can generate the meta prompt 220 with the provided traffic scene information to generate a simulated trajectory for an identified traffic scene. The decision-making entity C 255 can then perform the simulated trajectory by controlling an autonomous vehicle based on the simulated trajectory. The autonomous vehicle can have an advanced driver assistance system (ADAS) that can perform and generate the actions based on the simulated trajectory.

The present embodiments present a framework to utilize an LLM-based optimizer with chain-of-thought and meta learning mechanism to optimize the prompt. By doing so, the present embodiments enhanced the performance of LLMs in performing downstream tasks.

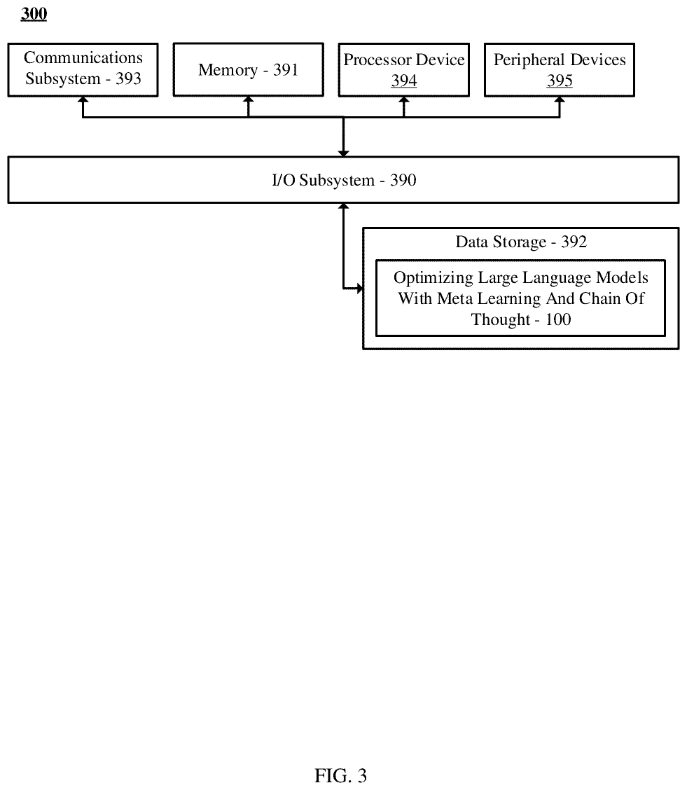

Referring now to FIG. 3, a block diagram showing a computing device implementing optimizing LLMs using meta learning and chain of thought, in accordance with an embodiment of the present invention.

The computing device 300 illustratively includes the processor device 394, an input/output (I/O) subsystem 390, a memory 391, a data storage device 392, and a communication subsystem 393, and/or other components and devices commonly found in a server or similar computing device. The computing device 300 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 391, or portions thereof, may be incorporated in the processor device 394 in some embodiments.

The processor device 394 may be embodied as any type of processor capable of performing the functions described herein. The processor device 394 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 391 may be embodied as any type of volatile or non-volatile memory or data storage capable of performing the functions described herein. In operation, the memory 391 may store various data and software employed during operation of the computing device 300, such as operating systems, applications, programs, libraries, and drivers. The memory 391 is communicatively coupled to the processor device 394 via the I/O subsystem 390, which may be embodied as circuitry and/or components to facilitate input/output operations with the processor device 394, the memory 391, and other components of the computing device 300. For example, the I/O subsystem 390 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 390 may form a portion of a system-on-a-chip (SOC) and be incorporated, along with the processor device 394, the memory 391, and other components of the computing device 300, on a single integrated circuit chip.

The data storage device 392 may be embodied as any type of device or devices configured for short-term or long-term storage of data such as, for example, memory devices and circuits, memory cards, hard disk drives, solid state drives, or other data storage devices. The data storage device 392 can store program code for optimizing large language models with meta learning and chain of thought 100. Any or all of these program code blocks may be included in a given computing system.

The communication subsystem 393 of the computing device 300 may be embodied as any network interface controller or other communication circuit, device, or collection thereof, capable of enabling communications between the computing device 300 and other remote devices over a network. The communication subsystem 393 may be configured to employ any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 300 may also include one or more peripheral devices 395. The peripheral devices 395 may include any number of additional input/output devices, interface devices, and/or other peripheral devices. For example, in some embodiments, the peripheral devices 395 may include a display, touch screen, graphics circuitry, keyboard, mouse, speaker system, microphone, network interface, and/or other input/output devices, interface devices, GPS, camera, and/or other peripheral devices.

Of course, the computing device 300 may also include other elements (not shown), as readily contemplated by one of skill in the art, as well as omit certain elements. For example, various other sensors, input devices, and/or output devices can be included in computing device 300, depending upon the particular implementation of the same, as readily understood by one of ordinary skill in the art. For example, various types of wireless and/or wired input and/or output devices can be employed. Moreover, additional processors, controllers, memories, and so forth, in various configurations can also be utilized. These and other variations of the computing device 300 are readily contemplated by one of ordinary skill in the art given the teachings of the present invention provided herein.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

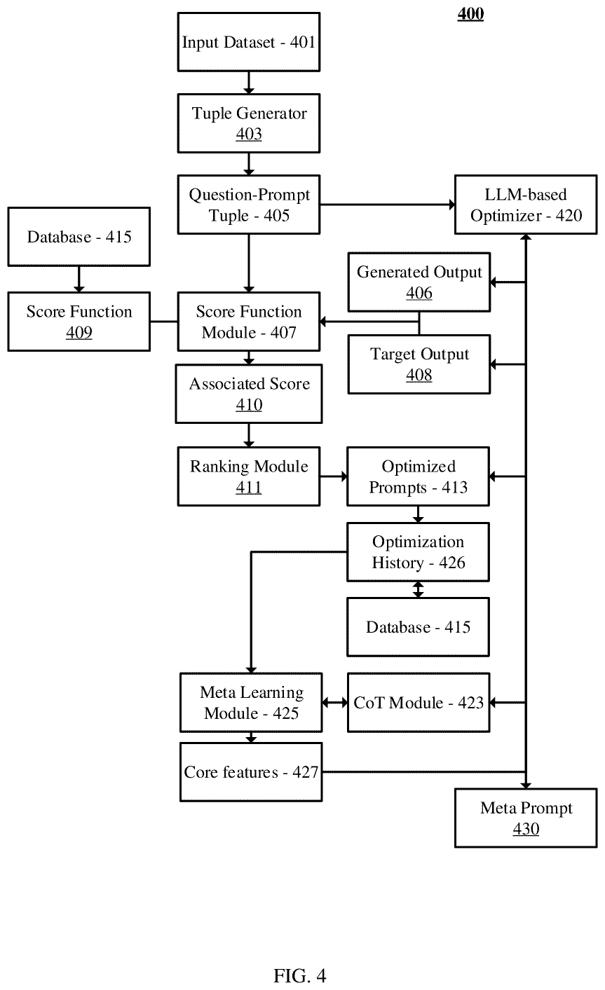

Referring now to FIG. 4, a block diagram showing hardware and software components employed to perform the operations in optimizing large language models with meta learning and chain of thought, in accordance with an embodiment of the present invention.

In system 400, an input dataset 401 can be processed by a tuple generator 403 to generate a question-prompt tuple 405 where the prompt is optimized and the question remains fixed. The LLM-based optimizer 420 can be utilized to generate generated output 406 and target output 408 based on the question-prompt tuple 405. A score function module 407 can compute an associated score 410 based on the generated output 406 and target output 408 by using a score function 409 obtained from a database 415 for the downstream task being performed. The associated score 410, the prompt from the question-prompt tuple 405 can be ranked by a ranking module 411 and the top ranked prompt from the question-prompt tuple 405 based on its associated score 410 can be included in the optimized prompts 413. The optimized prompts 413 and optimization history 426 can be fed into the meta learning module 425 which utilizes the chain of thought (CoT) module 423 to learn core features 427 by using the LLM-based optimizer 420. Using the core features 427, the LLM-based optimizer 420 can generate the meta prompt 430.

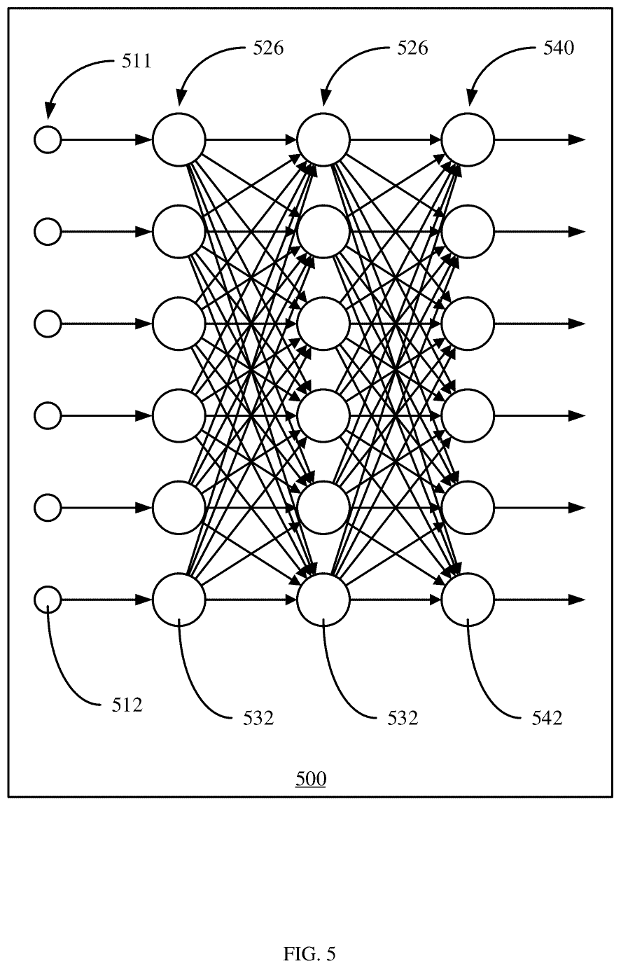

Referring now to FIG. 5, a block diagram showing a structure of deep neural networks for optimizing large language models with meta learning and chain of thought, in accordance with an embodiment of the present invention.

A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the inputted data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types and may include multiple distinct values. The network can have one input neurons for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

The deep neural network 500, such as a multilayer perceptron, can have an input layer 511 of source neurons 512, one or more computation layer(s) 526 having one or more computation neurons 532, and an output layer 540, where there is a single output neuron 542 for each possible category into which the input example could be classified. An input layer 511 can have a number of source neurons 512 equal to the number of data values 512 in the input data 511. The computation neurons 532 in the computation layer(s) 526 can also be referred to as hidden layers, because they are between the source neurons 512 and output neuron(s) 542 and are not directly observed. Each neuron 532, 542 in a computation layer generates a linear combination of weighted values from the values output from the neurons in a previous layer, and applies a non-linear activation function that is differentiable over the range of the linear combination. The weights applied to the value from each previous neuron can be denoted, for example, by w₁, w₂, . . . wₙ₋₁, wₙ. The output layer provides the overall response of the network to the inputted data. A deep neural network can be fully connected, where each neuron in a computational layer is connected to all other neurons in the previous layer, or may have other configurations of connections between layers. If links between neurons are missing, the network is referred to as partially connected.

In an embodiment, the computation layers 526 of the LLM-based optimizer 420 can learn relationships (e.g., core features, relevant information) between optimized prompts 413 by identify commonalities and differences between the optimized prompts 413. The output layer 540 of the LLM-based optimizer 420 can then provide the overall response of the network as a similarity score or difference score of the optimized prompts 413. In another embodiment, the LLM-based optimizer 420 can generate meta prompt based on the learned relationships.

Training a deep neural network can involve two phases, a forward phase where the weights of each neuron are fixed and the input propagates through the network, and a backwards phase where an error value is propagated backwards through the network and weight values are updated. The computation neurons 532 in the one or more computation (hidden) layer(s) 526 perform a nonlinear transformation on the input data 512 that generates a feature space. The classes or categories may be more easily separated in the feature space than in the original data space.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for optimizing artificial intelligence (AI) models, comprising:
fine-tuning a large language model (LLM) by generating optimized prompts based on an associated score of a generated output relative to a target output of a LLM-based optimizer using tuples of questions and prompts generated from a dataset;
learning core features from the dataset that obtained top-ranked associated scores for the optimized prompts by utilizing chain of thought mechanism; and
generating a meta prompt from the core features with the LLM-based optimizer to perform downstream tasks with the LLM.

2. The computer-implemented method of claim 1, further comprising updating a medical diagnosis of a patient using a specialized LLM configured for a downstream task with learned knowledge from healthcare data of the patient.

3. The computer-implemented method of claim 1, further comprising transferring learned knowledge to a specialized LLM by generalizing the meta prompt to new prompts for downstream tasks.

4. The computer-implemented method of claim 1, wherein the fine-tuning the LLM further comprises ranking the generated outputs based on the associated score.

5. The computer-implemented method of claim 3, wherein the fine-tuning the LLM further comprises generating a prompt function based on generalizations on tokens of the prompt performed by the LLM-based optimizer.

6. The computer-implemented method of claim 1, wherein learning the core features further comprises identifying commonalities from the optimized prompts by utilizing outputs of the chain of thought mechanism with a similarity computing function.

7. The computer-implemented method of claim 1, wherein learning the core features further comprises integrating differences from the optimized prompts by utilizing the outputs of the chain of thought mechanism into a learning process of the LLM-based optimizer.

8. A system for optimizing artificial intelligence (AI) models, comprising:
a memory device;
one or more processor devices operatively coupled with the memory device to:
fine-tune a large language model (LLM) by generating optimized prompts based on an associated score of a generated output relative to a target output of a LLM-based optimizer using tuples of questions and prompts generated from a dataset;
learn core features from the dataset that obtained top-ranked associated scores for the optimized prompts by utilizing chain of thought mechanism; and
generate a meta prompt from the core features with the LLM-based optimizer to perform downstream tasks with the LLM.

9. The system of claim 8, further comprising to update a medical diagnosis of a patient using a specialized LLM configured for a downstream task with learned knowledge from healthcare data of the patient.

10. The system of claim 8, further comprising transferring learned knowledge to a specialized LLM by generalizing the meta prompt to new prompts for downstream tasks.

11. The system of claim 8, wherein to fine-tune the LLM further comprises to rank the generated outputs based on the associated score.

12. The system of claim 11, wherein to fine-tune the LLM further comprises to generate a prompt function based on generalizations on tokens of the prompt performed by the LLM-based optimizer.

13. The system of claim 8, wherein to learn the core features further comprises to identify commonalities from the optimized prompts by utilizing outputs of the chain of thought mechanism with a similarity computing function.

14. The system of claim 8, wherein to learn the core features further comprises to integrate differences from the optimized prompts by utilizing the outputs of the chain of thought mechanism into a learning process of the LLM-based optimizer.

15. A non-transitory computer program product comprising a computer readable storage medium including program code for optimizing artificial intelligence (AI) models, wherein the program code when executed on a computer causes the computer to:
fine-tune a large language model (LLM) by generating optimized prompts based on an associated score of a generated output relative to a target output of a LLM-based optimizer using tuples of questions and prompts generated from a dataset;
learn core features from the dataset that obtained top-ranked associated scores for the optimized prompts by utilizing chain of thought mechanism; and
generate a meta prompt from the core features with the LLM-based optimizer to perform downstream tasks with the LLM.

16. The non-transitory computer program product of claim 15, further comprising to update a medical diagnosis of a patient using a specialized LLM configured for a downstream task with learned knowledge from healthcare data of the patient.

17. The non-transitory computer program product of claim 15, further comprising transferring learned knowledge to a specialized LLM by generalizing the meta prompt to new prompts for downstream tasks.

18. The non-transitory computer program product of claim 15, wherein to fine-tune the LLM further comprises to generate a prompt function based on generalizations on tokens of the prompt performed by the LLM-based optimizer.

19. The non-transitory computer program product of claim 15, wherein to learn the core features further comprises to identify commonalities from the optimized prompts by utilizing outputs of the chain of thought mechanism with a similarity computing function.

20. The non-transitory computer program product of claim 15, wherein to learn the core features further comprises to integrate differences from the optimized prompts by utilizing the outputs of the chain of thought mechanism into a learning process of the LLM-based optimizer.
